(19) United States
(12) Patent Application Publication
(10) Pub. No.: US 2026/0256852 A1
(43) Pub. Date: Sep. 3, 2026

(54) HYDROGELS CONTAINING ALDEHYDE GROUPS FOR CONTROLLED RELEASE OF BACTERIOPHAGES AND ANTIBIOTICS TO TREAT INFECTIONS

(71) Applicant: The Board of Trustees of the Leland Stanford Junior University, Stanford, CA (US)

(72) Inventors: Ovijit Chaudhuri, Stanford, CA (US); Paul L. Bollyky, Stanford, CA (US); Yung-Hao Lin, Stanford, CA (US); Qingquan Chen, Stanford, CA (US); Tejas Dharmaraj, Stanford, CA (US)

(73) Assignee: The Board of Trustees of the Leland Stanford Junior University, Stanford, CA (US)

(21) Appl. No.: 19/163,176

(22) PCT Filed: Mar. 14, 2024
(86) PCT No.: PCT/US2024/019951

§ 371 Date: Sep. 8, 2025

Related U.S. Application Data
(60) Provisional application No. 63/490,114, filed on Mar. 14, 2023.
(60) Provisional application No. 63/490,742, filed on Mar. 16, 2023.

(51) Int. Cl. A61K 35/76; A61K 9/06; A61K 47/10; A61K 47/34; A61K 47/36; A61K 47/69

(52) U.S. Cl. CPC A61K 35/76; A61K 9/06; A61K 47/10; A61K 47/34; A61K 47/36; A61K 47/6903

(57) ABSTRACT

Provided herein are hydrogels that include a plurality of amine-containing antimicrobial agents within the interiors of the hydrogels. The antimicrobial agents are connected to the hydrogel interiors via dynamic covalent imine bonds, and the hydrogel is engineered to facilitate controlled release of the antimicrobial agents. Also provided are methods for forming the provided hydrogels, and for using the hydrogels to treat a subject suffering from a microbial infection.

### CROSS-REFERENCE TO RELATED APPLICATIONS

The present application claims priority to U.S. Provisional Application No. 63/490,114, filed Mar. 14, 2023, and U.S. Provisional Application No. 63/490,742, filed Mar. 16, 2023, the full disclosures of which are incorporated herein by reference in their entirety for all purposes.

### BACKGROUND

Multi-drug resistant infections have emerged as one of the major health challenges worldwide, including in the context of orthopedic joint infections, wound infections, ear infections (chronic suppurative otitis media), and osteomyelitis. Because antibiotics, the current standard of care, are often by themselves insufficient to treat these infections, new approaches are needed.

Treatment with lytic bacteriophages (a.k.a., phages) provides a promising alternative approach to chronic antibiotic resistant biofilm infections. Phages are viruses that infect bacteria, multiply, and then cause lysis and bacterial death. However, the current method of sustained, intravenous delivery of active bacteriophages over a time period from days to weeks can be costly and inefficient.

### BRIEF SUMMARY

In one aspect, the disclosure provides a hydrogel including a plurality of amine-containing antimicrobial agents located within the interior of the hydrogel. Each of at least a portion of the amine-containing antimicrobial agents is connected to the hydrogel via one or more dynamic covalent imine bonds. The hydrogel is engineered to facilitate a controlled release of the amine-containing antimicrobial agents from the interior of the hydrogel.

In another aspect, the disclosure provides a method for treating a patient suffering from a microbial infection. The method includes administering to the patient a therapeutically effective amount of any of the hydrogels disclosed herein.

### BRIEF DESCRIPTION OF THE DRAWINGS

FIG. 1 presents an illustration of bacteriophage release from a hydrogel mediated by release of an imine bond between phage and hydrogel (i.e., dynamic covalent crosslink).

FIG. 2 presents an illustration of a hemithioacetal and thioether crosslinked hydrogel in accordance with a provided embodiment.

FIG. 3 Left: presents a photograph of a hemithioacetal and thioether dual-crosslinked hydrogel having 4 mg/mL thiolated hyaluronic acid (HA-Thiol), 1.25 mg/mL PEG-diacrylate (PEG-DA), and 20 mg/mL 4-arm PEG-aldehyde (4ALD). Right: presents a photograph of a hemithioacetal crosslinked hydrogel having 7 mg/mL HA-Thiol and 70 mg/mL 4ALD. These gels are formed under mild conditions without toxic chemicals or radiation, and are injectable, hydrolytically and enzymatically degradable, and self-healing.

FIG. 4 presents an illustration of an imine crosslinked hydrogel in accordance with a provided embodiment.

FIG. 5 presents photographs of an imine crosslinked hydrogel having 2% ε-poly-L-lysine (ε-PLL) and 17 mM 4ALD after 1 h of gelation. The imine gel is formed under mild conditions without toxic chemicals or radiation, and is injectable, hydrolytically degradable, and self-healing.

FIG. 6 presents an illustration of an ionic crosslinked aldehyde-containing hydrogel in accordance with a provided embodiment. The ionic crosslinked gel is formed under mild conditions without toxic chemicals or radiation, and is injectable and hydrolytically degradable.

FIG. 7 presents an illustration of a controlled release hydrogel using dynamic covalent crosslinking to deliver amine-containing antibiotics, an antimicrobial enzyme, or a peptide, in accordance with a provided embodiment.

FIG. 8 presents the rheological measurement of hemithioacetal and/or thioether crosslinked hydrogel, with the curves showing the storage modulus of the hydrogels evolves over time. The hemithioacetal crosslinked hydrogel that included 5 mg/ml of HA-Thiol and 50 mg/mL of 4ALD (bottom curve) did not form a gel, but a robust gel was formed with 10 mg/mL of HA-Thiol and 100 mg/mL of 4ALD (top curve). The thioether crosslinked hydrogel included 5 mg/mL of HA-Thiol and 0.625 mg/ml of PEG-DA (second curve from bottom). The hemithioacetal and thioether crosslinked hydrogel having 5 mg/mL of HA-Thiol, 0.625 mg/mL of PEG-DA, and 50 mg/mL of 4ALD (second curve from top), shows enhanced stiffness compared to hemithioacetal or thioether crosslinking alone.

FIG. 9 presents the gelation timing of various hemithioacetal and/or thioether crosslinked hydrogel formulations.

FIG. 10 presents the rheological measurement of imine crosslinked hydrogel with curves showing that the storage modulus of the hydrogels evolves over time. Higher ε-PLL or 4ALD concentration leads to faster gelation and higher storage modulus.

FIG. 11 presents the gelation timing of various imine crosslinked hydrogel formulations.

FIG. 12 shows that the hydrazone crosslinked alginate hydrogels immersed in fluid release bacteriophages in a sustained manner with tunable rate via modulation of the aldehyde to benzaldehyde ratio (i.e., the ratio of 4-arm PEG aldehyde or 4ALD to 4-arm PEG benzaldehyde or 4BLD).

FIG. 13 shows that the imine crosslinked hydrogels immersed in fluid release bacteriophages in a sustained manner. The polymer is epsilon poly-L-lysine and is crosslinked by 4ALD. The release rate is tunable via hydrogel component concentrations.

FIG. 14 shows that the imine crosslinked hydrogel in contact with minimal fluid release bacteriophages in a sustained manner. The polymer is epsilon poly-L-lysine and is crosslinked by 4ALD and 4BLD. The release rate is tunable via benzaldehyde ratio.

FIG. 15 presents a pair of graphs showing that the hemithioacetal and thioether crosslinked hydrogel in contact with minimal fluid presents sustained release of various strains of bacteriophage (e.g., PAML-31-1 and Luz24 phages, both targeting Pseudomonas aeruginosa bacteria). The encapsulated titer of phage was 1×10¹¹ PFU/mL in hydrogel.

FIG. 16 presents a pair of graphs showing that the hemithioacetal and thioether crosslinked hydrogel in contact with minimal fluid presents encapsulated phage titer dependent release kinetics. Higher titer of phages in the hydrogel led to higher release percentage of encapsulation.

FIG. 17 presents a graph plotting rheological measurements of hemithioacetal and/or thioether crosslinked hydrogel, with the plotted curves showing that the storage modulus of the hydrogels evolves over time. The thioether crosslinked hydrogel included 5 mg/mL of HA-Thiol and 1 mg/mL of HB-PEG (lower curve). The hemithioacetal and thioether crosslinked hydrogel included 5 mg/mL of GLYCOSIL®, 1 mg/mL of HB-PEG, and 75 mg/mL of 4-arm PEGs, 10% of which was 4BLD and 90% was 4ALD (upper curve). Addition of 4ALD and 4BLD crosslinker aids with faster increase of modulus and provides the sticky texture of the hydrogel.

### DETAILED DESCRIPTION

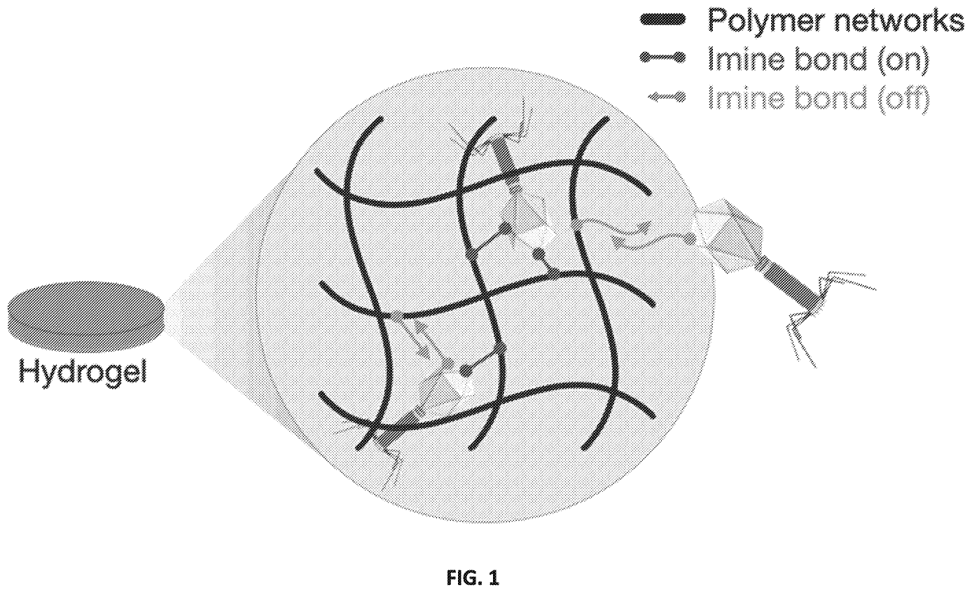

The present disclosure generally relates to aldehyde-containing hydrogels that release bacteriophage alone, or in combination with antibiotics, in a sustained manner over a relatively long period of time (e.g., days to weeks). The hydrogels are therefore particularly effective in treating infections, e.g., infections with a microbe targeted by the bacteriophage or antibiotic (FIG. 1). Various different hydrogel formulations for release of a bacteriophage or an antibiotic are provided.

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

As used herein, the singular forms “a,” “an,” and “the” include plural referents unless the content clearly dictates otherwise. Thus, for example, reference to “a thiolated anionic polysaccharide” optionally includes a combination of two or more thiolated anionic polysaccharides, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “and/or” refers to and encompasses any and all possible combinations of one or more of the associated listed items, as well as the lack of combinations when interpreted in the alternative (“or”).

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the terms “treat,” “treating,” and “treatment” refer to a procedure resulting in any indicia of success in the elimination or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of one or more symptoms. The treatment or amelioration of symptoms can be based on any objective or subjective parameter, including, e.g., the result of a physical examination or laboratory test.

As used herein, the term “therapeutically effective amount” refers to an amount or dose that produces the therapeutic effects for which it is administered. The exact amount or dose will depend on the purpose of the treatment and will be ascertainable by one skilled in the art using known techniques.

As used herein, the term “administering” refers to oral administration, administration as a suppository, topical contact, parenteral, intravenous, intraperitoneal, intramuscular, intralesional, intranasal, subcutaneous, intrathecal, intracerebroventricular, intraparenchymal, subretinal, or intravitreal administration to the subject.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

In one aspect, the present disclosure provides various hydrogels that generally include a plurality of amine-containing antimicrobial agents located within the interior of the hydrogel. In some examples, the antimicrobial agents include or consist of a plurality of bacteriophages. In some examples, the antimicrobial agents include or consist of a plurality of antibiotics. In some examples, the antimicrobial agents include or consist of a plurality of antimicrobial enzymes. In some examples, the antimicrobial agents include or consist of a plurality of antimicrobial peptides. The antimicrobial agents can alternatively include any combination of different types of antimicrobial agents, e.g., any combination of one or more species of bacteriophages, one or more species of antibiotics, one or more species of antimicrobial enzymes, and/or one or more species of antimicrobial peptides.

In some embodiments, the plurality of amine-containing antimicrobial agents in the provided hydrogel includes a plurality of at least one species of bacteriophage. The concentration of the bacteriophages in the hydrogel can be, for example, between about 10⁹ PFU/mL and about 10¹³ PFU/mL, e.g., between about 10⁹ PFU/mL and about 2.5×10¹¹ PFU/mL, between about 2.5×10⁹ PFU/mL and about 6.3×10¹¹ PFU/mL, between about 6.3×10⁹ PFU/mL and about 1.6×10¹² PFU/mL, between about 1.6×10¹⁰ PFU/mL and about 4×10¹² PFU/mL, or between about 4×10¹⁰ PFU/mL, and about 10¹³ PFU/mL. In terms of upper limits, the concentrations of the bacteriophages in the hydrogel can be, for example, no more than about 10¹³ PFU/mL, e.g., no more than about 4×10¹² PFU/mL, no more than about 1.6×10¹² PFU/mL, no more than about 6.3×10¹¹ PFU/mL, no more than about 2.5×10¹¹ PFU/mL, no more than about 10¹¹ PFU/mL, no more than about 4×10¹⁰ PFU/mL, no more than about 1.6×10¹⁰ PFU/mL, no more than about 6.3×10⁹ PFU/mL, or no more than about 2.5×10⁹ PFU/mL. In terms of lower limits, the concentration of the bacteriophages in the hydrogel can be, for example, no less than about 10⁹ PFU/mL, e.g., no less than about 2.5×10⁹ PFU/mL, no less than about 6.3×10⁹ PFU/mL, no less than about 1.6×10⁹ PFU/mL, no less than about 4×10¹⁰ PFU/mL, no less than about 10¹¹ PFU/mL, no less than about 2.5×10¹¹ PFU/mL, no less than about 6.3×10¹¹ PFU/mL, no less than about 1.6×10¹² PFU/mL, or no less than about 4×10¹¹ PFU/mL.

Each of at least a portion of the plurality of amine-containing antimicrobial agents in the provided hydrogel can be connected to the hydrogel by one or more dynamic covalent bonds, e.g., one or more dynamic covalent imine bonds. The hydrogel can be configured, i.e., engineered, to facilitate a controlled release of the antimicrobial agents from the interior of the hydrogel. In some embodiments, the controlled release is facilitated by the release, i.e., unbinding, of the dynamic covalent bonds.

In some embodiments, the hydrogel includes a thiolated anionic polysaccharide, e.g., thiolated hyaluronic acid. The concentration of the thiolated anionic polysaccharide in the hydrogel can be, for example, between about 1.5 mg/mL and about 15 mg/mL, e.g., between about 1.5 mg/mL and about 9.6 mg/mL, between about 2.9 mg/mL and about 11 mg/mL, between about 4.2 mg/mL and about 12.3 mg/mL, between about 5.6 mg/mL and about 13.7 mg/mL, or between about 6.9 mg/mL and about 15 mg/mL. In terms of upper limits, the concentration of the thiolated anionic polysaccharide in the hydrogel can be, for example, no more than about 15 mg/ml, e.g., no more than about 13.65 mg/mL, no more than about 12.3 mg/mL, no more than about 11 mg/mL, no more than about 9.6 mg/mL, no more than about 8.3 mg/mL, no more than about 6.9 mg/mL, no more than about 5.6 mg/mL, no more than about 4.2 mg/mL, or no more than about 2.9 mg/mL. In terms of lower limits, the concentration of the thiolated anionic polysaccharide in the hydrogel can be, for example, no less than about 1.5 mg/mL, e.g., no less than about 2.9 mg/mL, no less than about 4.2 mg/mL, no less than about 5.6 mg/mL, no less than about 6.9 mg/mL, no less than about 8.3 mg/mL, no less than about 9.6 mg/mL, no less than about 11 mg/mL, no less than about 12.3 mg/mL, or no less than about 13.7 mg/mL.

In some embodiments, the hydrogel includes an aldehyde-functionalized crosslinker, e.g., a multi-arm PEG-aldehyde such as PEG-dialdehyde, 4-arm PEG-aldehyde, or a combination thereof. The concentration of the aldehyde-functionalized crosslinker in the hydrogel can be, for example, between about 22.5 mg/mL and about 225 mg/mL, e.g., between about 22.5 mg/mL and about 144 mg/mL, between about 42.8 mg/mL and about 164 mg/mL, between about 63 mg/mL and about 185 mg/mL, between about 83.2 mg/mL and about 205 mg/mL, or between about 104 mg/mL and about 225 mg/mL. In terms of upper limits, the concentration of the aldehyde-functionalized crosslinker in the hydrogel can be, for example, no more than about 225 mg/ml, e.g., no more than about 205 mg/mL, no more than about 185 mg/mL, no more than about 164 mg/mL, no more than about 144 mg/mL, no more than about 124 mg/mL, no more than about 104 mg/mL, no more than about 83.2 mg/mL, no more than about 63 mg/mL, or no more than about 42.8 mg/mL. In terms of lower limits, the concentration of the aldehyde-functionalized crosslinker in the hydrogel can be, for example, no less than about 22.5 mg/mL, e.g., no less than about 42.8 mg/mL, no less than about 63 mg/mL, no less than about 83.3 mg/mL, no less than about 103 mg/mL, no less than about 124 mg/mL, no less than about 144 mg/mL, no less than about 164 mg/mL, no less than about 185 mg/mL, or no less than about 205 mg/mL.

In some embodiments, the hydrogel includes a benzaldehyde-functionalized crosslinker, e.g., a multi-arm PEG-benzaldehyde such as PEG-dibenzaldehyde, 4-arm PEG-benzaldehyde, or a combination thereof. The concentration of the benzaldehyde-functionalized crosslinker in the hydrogel can be, for example, between about 2.5 mg/mL and about 25 mg/mL, e.g., between about 2.5 mg/mL and about 16 mg/mL, between about 4.75 mg/mL and about 18.25 mg/mL, between about 7 mg/mL and about 20.5 mg/mL, between about 9.25 mg/mL and about 22.75 mg/mL, or between about 11.5 mg/mL and about 25 mg/mL. In terms of upper limits, the concentration of the benzaldehyde-functionalized crosslinker in the hydrogel can be, for example, no more than about 25 mg/ml, e.g., no more than about 22.75 mg/mL, no more than about 20.5 mg/mL, no more than about 18.25 mg/mL, no more than about 16 mg/mL, no more than about 13.75 mg/mL, no more than about 11.5 mg/mL, no more than about 9.25 mg/mL, no more than about 7 mg/mL, or no more than about 4.75 mg/mL. In terms of lower limits, the concentration of the benzaldehyde-functionalized crosslinker in the hydrogel can be, for example, no less than about 2.5 mg/mL, e.g., no less than about 4.75 mg/mL, no less than about 7 mg/mL, no less than about 9.25 mg/mL, no less than about 11.5 mg/mL, no less than about 13.75 mg/mL, no less than about 16 mg/mL, no less than about 18.25 mg/mL, no less than about 20.5 mg/mL, or no less than about 22.75 mg/mL.

The relative concentrations between an aldehyde-functionalized crosslinker and a benzaldehyde-functionalized crosslinker in the provided hydrogel can be selected or configured to provide the hydrogel with desired rates of release of the amine-containing antimicrobial agents from the hydrogel. For example, the mass ratio of the aldehyde-functionalized crosslinker to the benzaldehyde-functionalized crosslinker in the hydrogel can be at least about 5:1, e.g., at least about 7:1, at least about 10:1, at least about 15:1, at least about 20:1, at least about 30:1, at least about 45:1, at least about 65:1, at least about 95:1, at least about 140:1, or at least about 200:1. The mass ratio of the aldehyde-functionalized crosslinker to the benzaldehyde-functionalized crosslinker in the hydrogel can be, for example, between about 5:1 and about 200:1, e.g., between about 5:1 and about 45:1, between about 7:1 and about 65:1, between about 10:1 and about 95:1, between about 15:1 and about 140:1, or between about 20:1 and about 200:1.

In some embodiments, the hydrogel includes an acrylate-functionalized crosslinker, e.g., a multi-arm PEG-acrylate such as PEG-diacrylate, a hyperbranched PEG-acrylate, or a combination thereof. The concentration of the acrylate-functionalized crosslinker in the hydrogel can be, for example, between about 0.3 mg/mL and about 3 mg/mL, e.g., between about 0.3 mg/mL and about 1.9 mg/mL, between about 0.6 mg/mL and about 2.2 mg/mL, between about 0.8 mg/mL and about 2.5 mg/mL, between about 1.1 mg/mL and about 2.7 mg/mL, or between about 1.4 mg/mL and about 3 mg/mL. In terms of upper limits, the concentration of the acrylate-functionalized crosslinker in the hydrogel can be, for example, no more than about 3 mg/ml, e.g., no more than about 2.7 mg/mL, no more than about 2.5 mg/mL, no more than about 2.2 mg/mL, no more than about 1.9 mg/mL, no more than about 1.7 mg/mL, no more than about 1.4 mg/mL, no more than about 1.1 mg/mL, no more than about 0.8 mg/mL, or no more than about 0.6 mg/mL. In terms of lower limits, the concentration of the acrylate-functionalized crosslinker in the hydrogel can be, for example, no less than about 0.3 mg/mL, e.g., no less than about 0.6 mg/mL, no less than about 0.8 mg/mL, no less than about 1.1 mg/mL, no less than about 1.4 mg/mL, no less than about 1.7 mg/mL, no less than about 1.9 mg/mL, no less than about 2.2 mg/mL, no less than about 2.5 mg/mL, or no less than about 2.7 mg/mL.

In some embodiments, the hydrogel includes a polylysine, e.g., ε-poly-L-lysine. The concentration of the polylysine in the hydrogel can be, for example, between about 1 mg/mL and about 40 mg/mL, e.g., between about 1 mg/mL and about 9.1 mg/mL, between about 1.4 mg/mL and about 13 mg/mL, between about 2.1 mg/mL and about 19 mg/mL, between about 3 mg/mL and about 28 mg/mL, or between about 4.4 mg/mL and about 40 mg/mL. In terms of upper limits, the concentration of the polylysine in the hydrogel can be, for example, no more than about 40 mg/ml, e.g., no more than about 28 mg/mL, no more than about 19 mg/mL, no more than about 13 mg/mL, no more than about 9.1 mg/mL, no more than about 6.3 mg/mL, no more than about 4.4 mg/mL, no more than about 3 mg/mL, no more than about 2.1 mg/mL, or no more than about 1.4 mg/mL. In terms of lower limits, the concentration of the polylysine in the hydrogel can be, for example, no less than about 1 mg/mL, e.g., no less than about 1.4 mg/mL, no less than about 2.1 mg/mL, no less than about 3 mg/mL, no less than about 4.4 mg/mL, no less than about 6.3 mg/mL, no less than about 9.1 mg/mL, no less than about 13 mg/mL, no less than about 19 mg/mL, or no less than about 28 mg/mL.

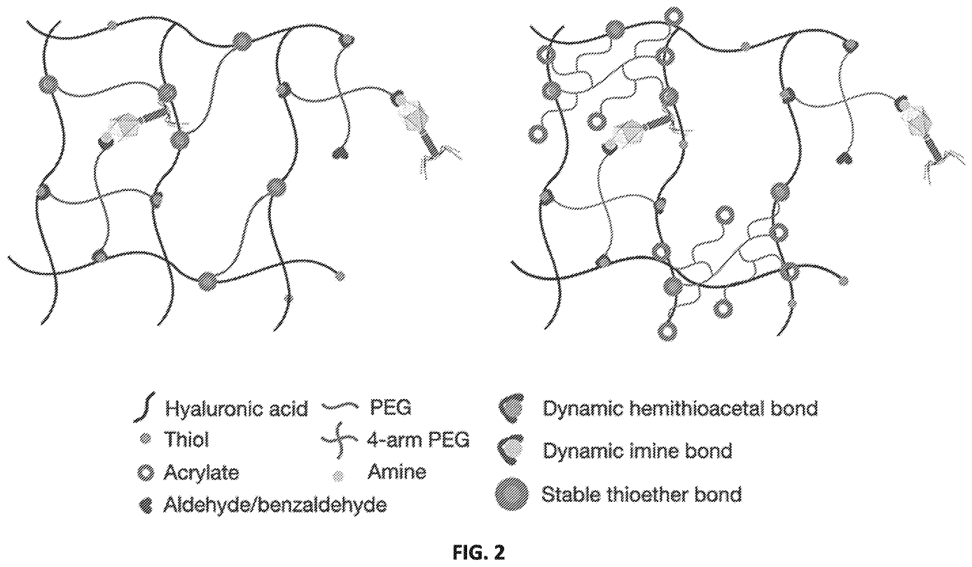

In some embodiments, and as shown in FIGS. 2 and 3, the provided controlled release hydrogel includes thiolated hyaluronic acid (hyaluronic acid polymer with a thiol group coupled to it), 4-arm PEG-aldehyde, 4-arm PEG-benzaldehyde, and bacteriophage. The 4-arm PEG-aldehyde and 4-arm PEG-benzaldehyde form dynamic hemithioacetal bonds to the hyaluronic acid, thereby forming a hydrogel. The 4-arm PEG-aldehyde and 4-arm PEG-benzaldehyde also form imine bonds with the primary amine groups on the bacteriophages. Therefore, phages are tethered to the alginate hydrogel through the 4-arm PEG crosslinkers. Release/unbinding of the dynamic covalent crosslinks (imine bonds) allows the phage to diffuse through the hydrogel and out of the hydrogel into the surrounding area over long times. In some embodiments, to better allow the dynamic hemithioacetal bonds to form a hydrogel that is stable over at timescale for a week, and that has a minimum initial elastic modulus of 0.1 kPa, a PEG-diacrylate or hyperbranched PEG-acrylate (HB-PEG) crosslinker is added. The acrylate groups form a stable thioether bond to the thiol groups on the hyaluronic acid. Such thioether bonds are known to form robust hydrogels.

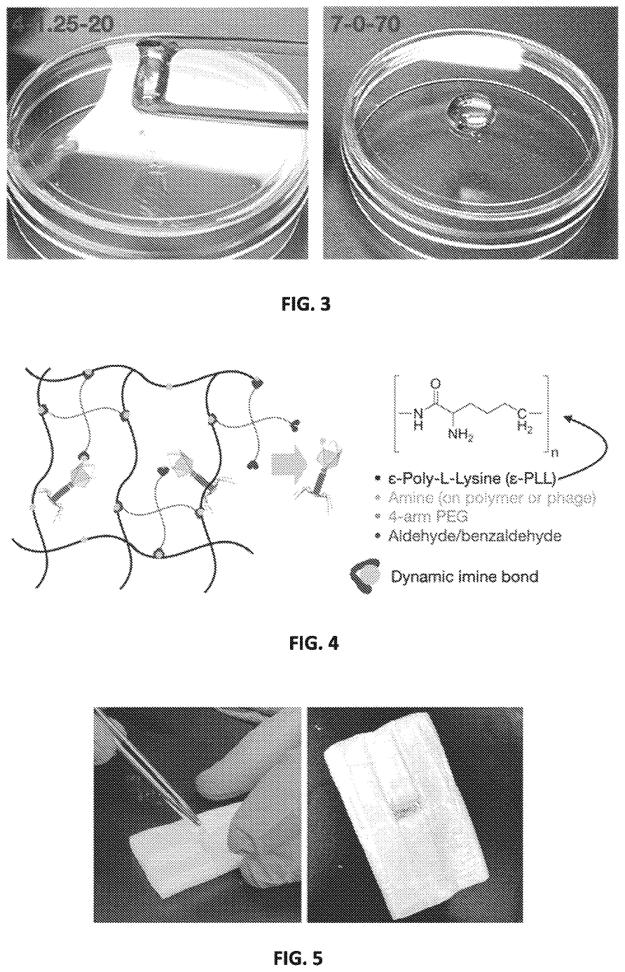

In some embodiments, and as shown in FIGS. 4 and 5, the provided controlled release hydrogel includes ε-poly-L-lysine, 4-arm PEG-aldehyde, 4-arm PEG-benzaldehyde, and bacteriophage. The 4-arm PEG-aldehyde and 4-arm-PEG-benzaldehyde form a dynamic imine bond with the lysine groups on the ε-poly-L-lysine polymer to form a hydrogel. The 4-arm PEG-aldehyde and 4-arm PEG-benzaldehyde also form imine bonds with the primary amine groups on the bacteriophages. Therefore, phages are tethered to the alginate hydrogel through the 4-arm PEG crosslinkers. Release/unbinding of the dynamic covalent crosslinks (imine bonds) allows the phage to diffuse through the hydrogel and out of the hydrogel into the surrounding area over long times. In some embodiments, to better allow the crosslinked ε-poly-L-lysine to form a sufficiently robust and stable hydrogel, an interpenetrating network of calcium crosslinked alginate is used to make the composite gel. In some such embodiments, the interpenetrating alginate network does not form any bonds with the 4-arm PEGs or the phage. In some embodiments, to better increase the viscosity of the crosslinked ε-poly-L-lysine, thickeners are used to make composite gel. Suitable thickeners include, but are not limited to, unmodified PEG, unmodified alginate, corn starch, dextran, methylcellulose, Carbopol, and combinations thereof. In some such embodiments, the thickeners do not form any bonds with the hydrogel polymer, 4-arm PEGs, or the phage.

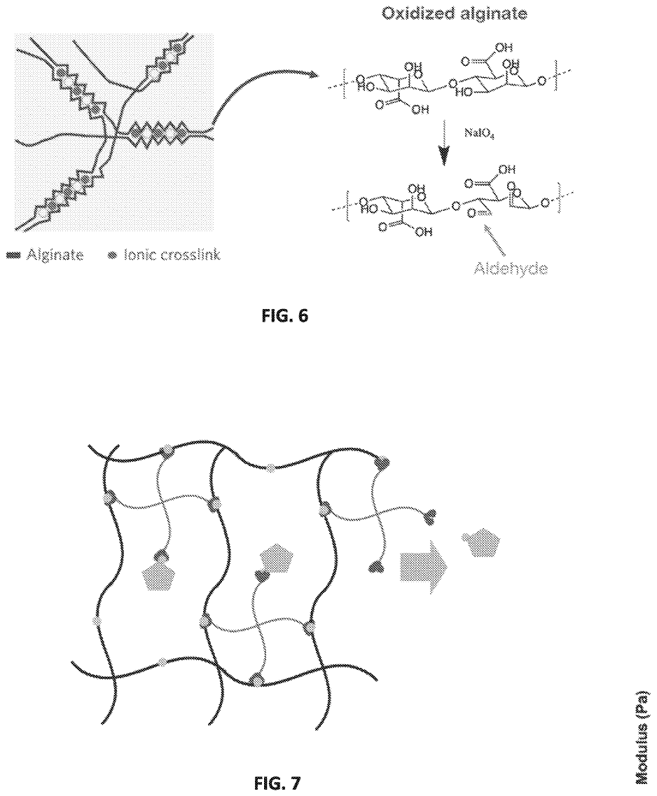

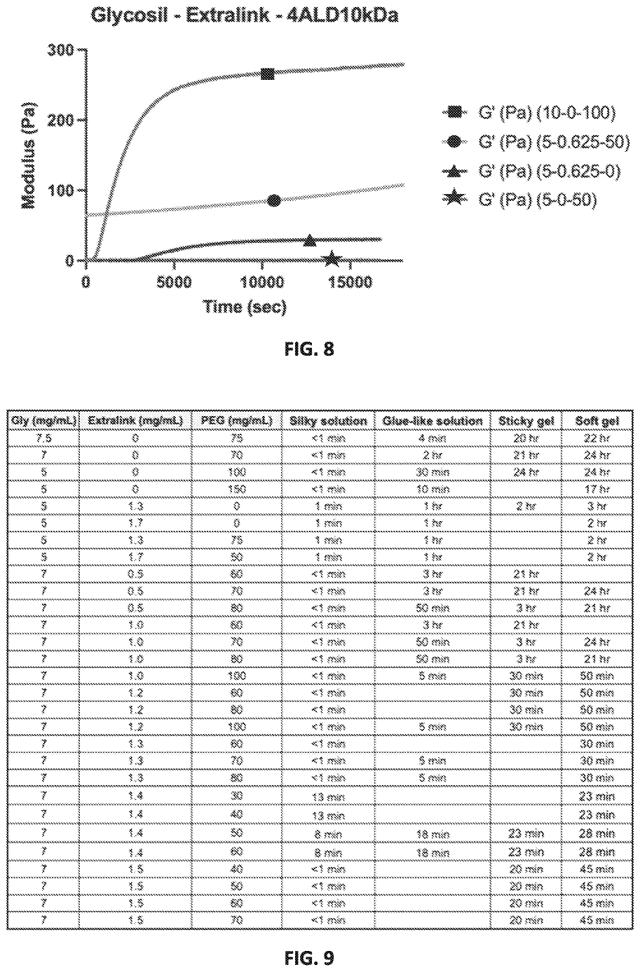

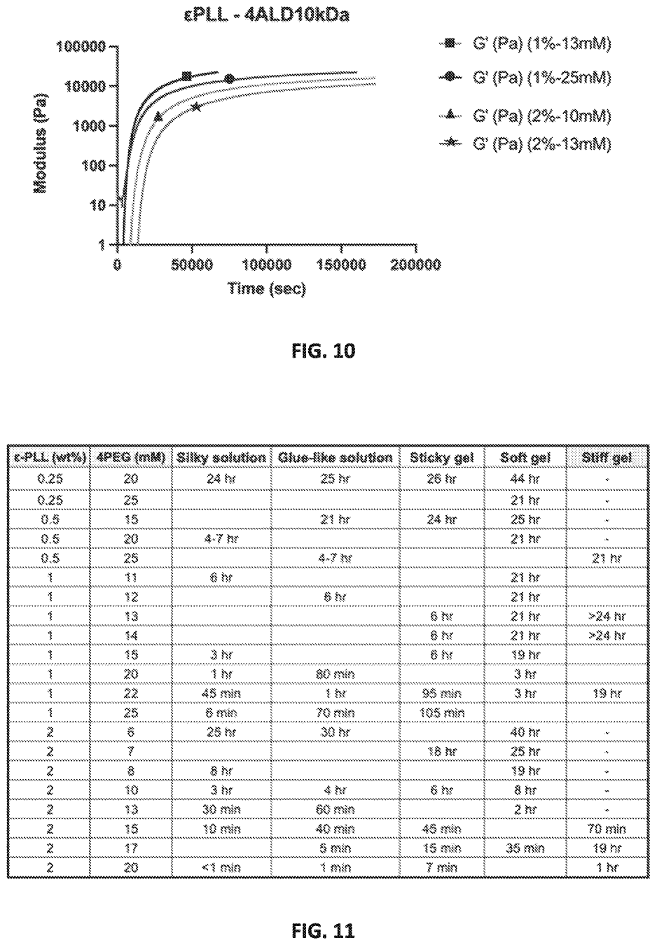

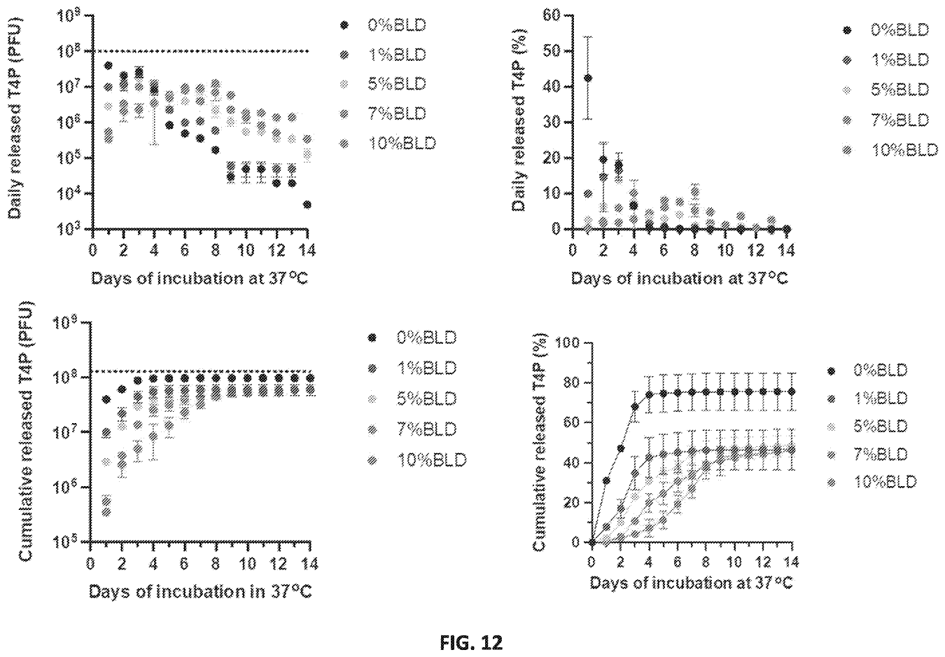

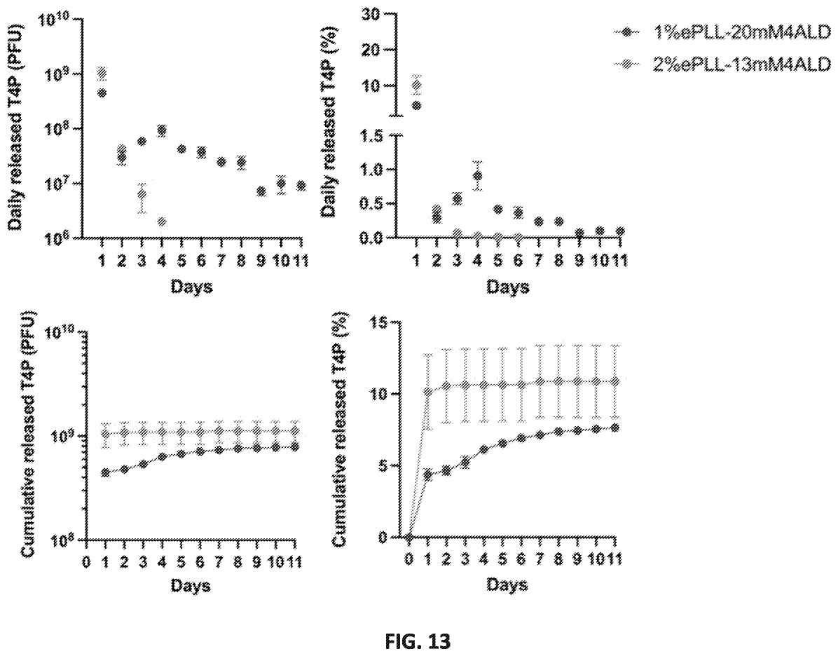

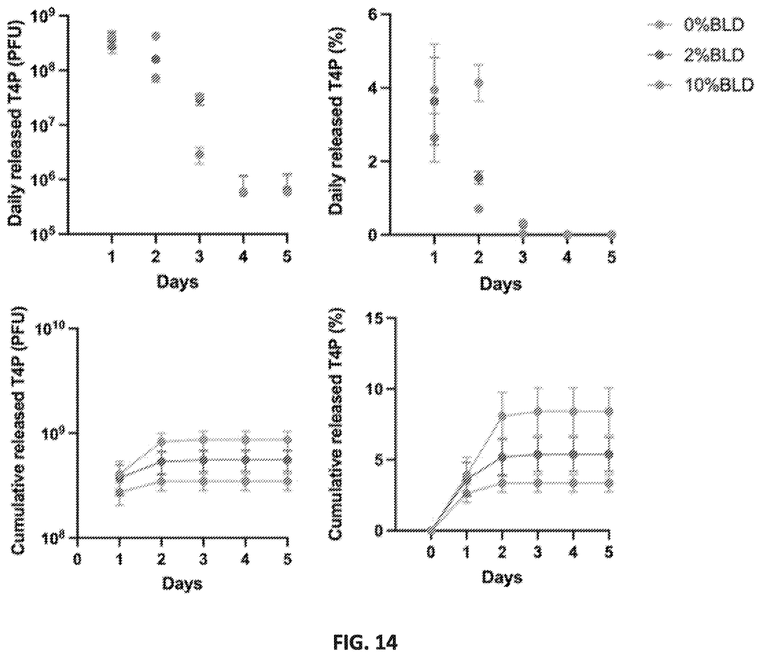

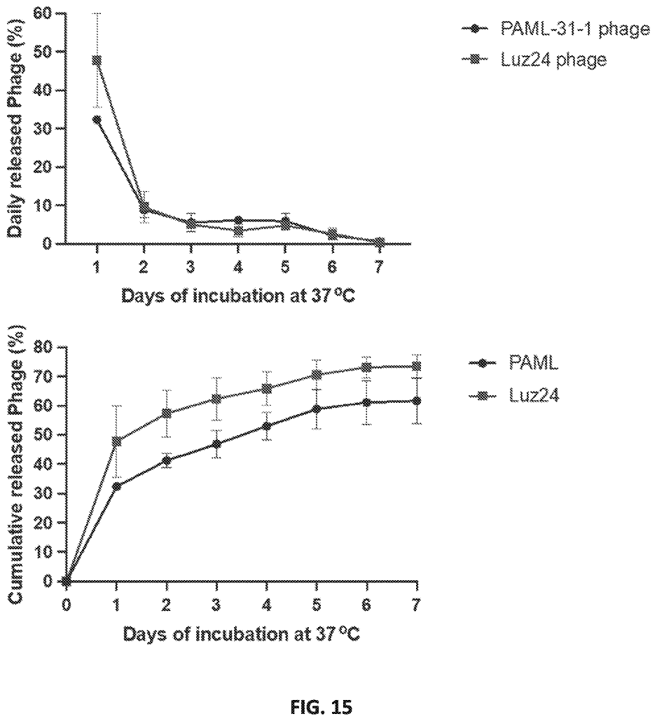

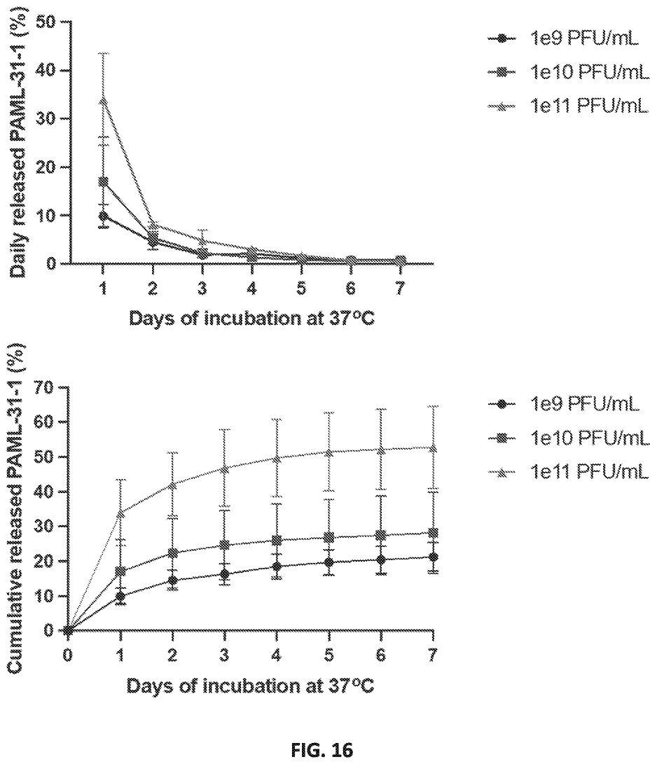

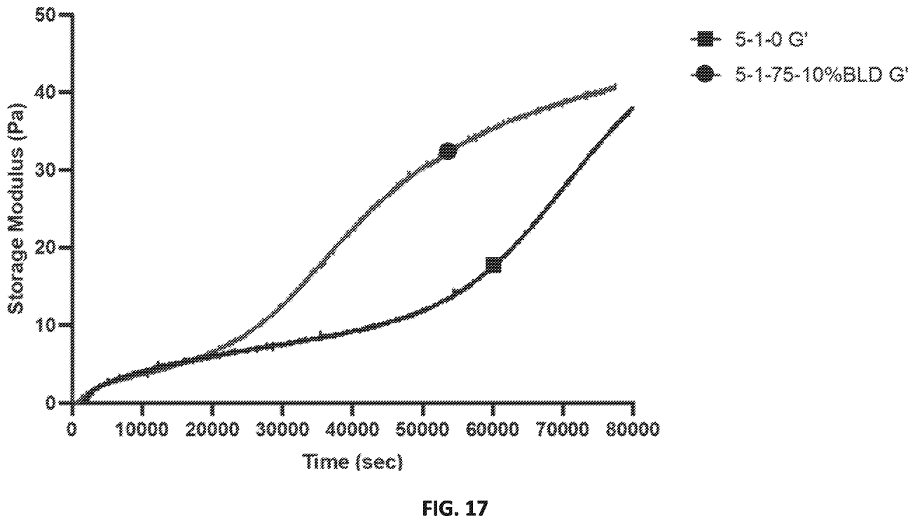

In some embodiments, phage is coupled to the provided controlled release hydrogel through a 4-arm-PEG-aldehyde and 4-arm PEG-benzaldehyde crosslinker. Different base polymers can be used in the hydrogel, leading to different types of bonds formed between the hydrogel polymer and the 4-arm PEG crosslinkers. In some embodiments, and as shown in FIG. 6, a hybrid gel is created where crosslinks forming the hydrogel polymer are ionic and the bonds between the hydrogel polymer and the phage are dynamic covalent bonds. Suitable compositions for forming the provided controlled release hydrogel include, but are not limited to:
1. Hyaluronic acid-amine+PEG aldehyde/PEG benzaldehyde+phage
2. Hyaluronic acid-hydrazide+PEG aldehyde/PEG benzaldehyde+phage
3. Hyaluronic acid-hydrazine+PEG aldehyde/PEG benzaldehyde+phage
4. PEG-hydrazide+PEG aldehyde/PEG benzaldehyde+phage
5. PEG-hydrazine+PEG aldehyde/PEG benzaldehyde+phage
6. Alginate-amine+PEG aldehyde/PEG benzaldehyde+phage
7. Alginate-hydrazide+PEG aldehyde/PEG benzaldehyde+phage
8. Alginate-hydrazine+PEG aldehyde/PEG benzaldehyde+phage
9. Poly-L-lysine+PEG aldehyde/PEG benzaldehyde+phage
10. Poly-D-Lysine+PEG aldehyde/PEG benzaldehyde+phage
11. Glycol-chitosan+PEG aldehyde/PEG benzaldehyde+phage
12. Alginate coupled to aldehyde and/or benzaldehyde groups in a well-defined ratio+divalent calcium crosslinker+phage
13. Hyaluronic acid coupled to aldehyde and/or benzaldehyde groups in a well-defined ratio+divalent calcium crosslinker+phage
14. Any of compositions 1-10 above, with an additional interpenetrating network hydrogel (e.g., ionically crosslinked alginate) to make the gel more stable and robust
15. Hybrid gel of oxidized alginate+divalent calcium crosslinker+phage
16. Hybrid gel of oxidized dextran+divalent calcium crosslinker+phage
17. Hybrid gel of oxidized chitosan+divalent calcium crosslinker+phage
18. Hybrid gel of oxidized Hyaluronic acid+divalent calcium crosslinker+phage
   In some embodiments, one or both of the 4-arm PEG aldehyde and 4-arm PEG benzaldehyde in any of the above compositions can be substituted with PEG dialdehyde, PEG dibenzaldehyde, multi-arm PEG aldehyde, multi-arm PEG benzaldehyde, or any combination thereof.

In some embodiments, the aldehyde to benzaldehyde group ratio in the provided controlled release hydrogel is modulated to tune the kinetics of phage release to a desired timeline.

In some embodiments, and as shown in FIG. 7, the provided controlled release hydrogels are formed in the presence of antibiotics to also allow release of antibiotics to treat the infection in parallel with the bacteriophage. For example, amine-containing antibiotics can be included in any of the above hydrogels to effect long and sustained release of the antibiotics.

In some embodiments, the provided controlled release hydrogel is administered to a subject to treat a joint implant infection. In some embodiments, the controlled release hydrogel is administered to a subject to treat a wound infection. In some embodiments, the controlled release hydrogel is administered to a subject to treat an ear infection. In some embodiments, the controlled release hydrogel is administered to a subject to treat chronic suppurative otitis media. In some embodiments, the controlled release hydrogel is administered to a subject to treat osteomyelitis.

The range of bacteriophages that can be delivered with the provided controlled release hydrogels is broad. The hydrogels are suitable for delivering both enveloped and non-enveloped phages, and phages with DNA and RNA genomes. Phages suitable for delivery from the provided controlled release hydrogels include, but are not limited to, those from the following taxonomic groups: Belfryvirales, Turriviridae, Caudovirales, Ackermannviridae, Autographiviridae, Chaseviridae, Demerecviridae, Drexlerviridae, Guenliviridae, Herelleviridae, Myoviridae, Siphoviridae, Podoviridae, Rountreeviridae, Salasmaviridae Schitoviridae, Zobellviridae, Halopanivirales, Sphaerolipoviridae, Simuloviridae, Matshushitaviridae, Haloruvirae, Pleolipoviridae, Kalamavirales, Tectiviridae, Ligamenvirales, Lipothrixviridae, Acidianus Rudiviridae, Mindivirales, Cystoviridae, Atkinsviridae, Duinviridae, Fiersviridae, Solspiviridae, Petitvirales, Microviridae, Primavirales, Tristromavirida, Timlovirales, Blumeviridae, Steitzviridae, Tubulavirales, Inoviridae, Paulinoviridae, Plectroviridae, Vinavirales, Corticoviridae, Dumavirales Picobimaviridae, Ampullaviridae, Autolykiviridae, Bicaudaviridae, Globuloviridae, Guttaviridae, Halspiviridae, Plasmaviridae, Portogloboviridae, Thaspiviridae, and Spiraviridae.

The provided controlled release hydrogels can also be used to deliver viral particles made by fungi (mycophages) or eukaryotic cells (viruses). These include, but are not limited to, for example, Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Bimaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae Fuselloviridae, Gammaflexiviridae, Geminiviridae, Genomoviridae, Globuloviridae, Gresnaviridae, Quelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Mamaviridae, Marseilleviridae, Matonaviridae, Matshushitaviridae, Mayoviridae, Medioniviridae, Megabimaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Namaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobimaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, and Zobellviridae.

Modified or engineered bacteriophages can also be suitable for use with the provided controlled release hydrogels. These include phages stripped of bacterial endotoxin and/or lipopolysaccharide as well as phages altered to express different proteins, nucleotides, and/or lipids. Additional suitable modifications to phage included in the provided hydrogels include engineering of phage capsid proteins to deliver retinoids (e.g., Retin-a, etc.), β-hydroxy acid (salicylic acid), hydroquinone, kojic acid, L-ascorbic acid (vitamin C), glycosaminoglycans (e.g., hyaluronan, etc.), copper peptide, α-lipoic acid, DMAE (dimethylaminoethanol), hormones, growth factors, thickening agents (e.g., xanthan, carrageenan, alginate or chemically modified cellulose compounds), emulsifiers (e.g., isopropyl myristate (IPM) or diisopropyl adipate, polyacrylates, etc.), aromatic agents (e.g., anise alcohol, benzyl alcohol, coumarin, eugenol, hydroxycitronella, limonene and geraniol), α-hydroxy acids (glycolic, lactic, tartaric, citric acids, etc.), polyhydroxy acids, preservatives, antioxidants, pigments, other additives, or any combination thereof. Additionally, phages can be encapsulated in other materials and then embedded in the hydrogel (e.g., into microspheres, liposome, or microsomes with or without surface modifications that enable binding to the gel).

The range of antibiotics that can be delivered with the provided controlled release hydrogels is also broad. Antibiotics suitable for delivery from the provided controlled release hydrogels include, but are not limited to, those from classes including penicillins, fluoroquinolones, cephalosporins, macrolides, carbapenems, β-lactams with increased activity (e.g., amoxicillin-clavulanate), tetracyclines, trimethoprim-sulfamethoxazole, lincosamides (e.g., clindamycin), urinary anti-infectives, and other antibiotics including gentamicin. Other agents known to have anti-microbial properties can also be included in the provided hydrogels for delivery through controlled release. Modified or engineered antibiotics could also be included in the provided hydrogels. These can include, for example, antibiotics modified to reduce toxicity or to enable binding to the gel.

The following embodiments are contemplated. All combinations of features and embodiments are contemplated.

Embodiment 1: A hydrogel comprising a plurality of amine-containing antimicrobial agents located within the interior of the hydrogel, wherein each of at least a portion of the amine-containing antimicrobial agents is connected to the hydrogel via one or more dynamic covalent imine bonds, and wherein the hydrogel is engineered to facilitate a controlled release of the amine-containing antimicrobial agents from the interior of the hydrogel.

Embodiment 2: An embodiment of embodiment 1, wherein the plurality of amine-containing antimicrobial agents comprises a plurality of bacteriophages, a plurality of antibiotics, a plurality of antimicrobial enzymes, a plurality of antimicrobial peptides, or a combination thereof.

Embodiment 3: An embodiment of embodiment 2, wherein the plurality of amine-containing antimicrobial agents comprises the plurality of bacteriophages.

Embodiment 4: An embodiment of embodiment 3, wherein the concentration of the plurality of bacteriophages in the hydrogel is between 10⁹ PFU/mL and 10¹³ PFU/mL.

Embodiment 5: An embodiment of any one of embodiments 1-4, wherein the hydrogel is crosslinked with dynamic covalent hydrazone bonds, dynamic covalent hemithioacetal bonds, stable covalent thioether bonds, dynamic covalent imine bonds, ionic bonds, static covalent bonds, or a combination thereof.

Embodiment 6: An embodiment of embodiment 5, wherein the hydrogel is crosslinked with dynamic covalent hemithioacetal bonds, stable covalent thioether bonds, or a combination thereof.

Embodiment 7: An embodiment of any one of embodiments 1-6, wherein the hydrogel further comprises a thiolated anionic polysaccharide.

Embodiment 8: An embodiment of embodiment 7, wherein the thiolated anionic polysaccharide comprises thiolated hyaluronic acid.

Embodiment 9: An embodiment of embodiment 7 or 8, wherein the concentration of the thiolated anionic polysaccharide in the hydrogel is between 1.5 mg/mL and 15 mg/mL.

Embodiment 10: An embodiment of any one of embodiments 1-9, wherein the hydrogel further comprises an aldehyde-functionalized crosslinker.

Embodiment 11: An embodiment of embodiment 10, wherein the aldehyde-functionalized crosslinker comprises a multi-arm poly(ethylene glycol) (PEG)-aldehyde.

Embodiment 12: An embodiment of embodiment 11, wherein the multi-arm PEG-aldehyde comprises PEG-dialdehyde, 4-arm PEG-aldehyde, or a combination thereof.

Embodiment 13: An embodiment of any one of embodiments 10-12, wherein the concentration of the aldehyde-functionalized crosslinker in the hydrogel is between 22.5 mg/mL and 225 mg/mL.

Embodiment 14: An embodiment of any one of embodiments 1-13, wherein the hydrogel further comprises a benzaldehyde-functionalized crosslinker.

Embodiment 15: An embodiment of embodiment 14, wherein the benzaldehyde-functionalized crosslinker comprises a multi-arm PEG-benzaldehyde.

Embodiment 16: An embodiment of embodiment 15, wherein the multi-arm PEG-benzaldehyde comprises PEG-dibenzaldehyde, 4-arm PEG-benzaldehyde, or a combination thereof.

Embodiment 17: An embodiment of any one of embodiments 14-16, wherein the concentration of the benzaldehyde-functionalized crosslinker in the hydrogel is between 2.5 mg/mL and 25 mg/mL.

Embodiment 18: An embodiment of any one of embodiments 1-17, wherein the hydrogel comprises an aldehyde-functionalized crosslinker and a benzaldehyde-functionalized crosslinker, and wherein the mass ratio of the aldehyde-functionalized crosslinker to the benzaldehyde-functionalized crosslinker in the hydrogel is at least 5:1.

Embodiment 19: An embodiment of any one of embodiments 1-18, wherein the hydrogel further comprises an acrylate-functionalized crosslinker.

Embodiment 20: An embodiment of embodiment 19, wherein the acrylate-functionalized crosslinker comprises PEG diacrylate, a hyperbranched PEG acrylate, or a combination thereof.

Embodiment 21: An embodiment of embodiment 19 or 20, wherein the concentration of the acrylate-functionalized crosslinker in the hydrogel is between 0.3 mg/mL and 3 mg/mL.

Embodiment 22: An embodiment of any one of embodiments 1-21, wherein the hydrogel further comprises a polylysine.

Embodiment 23: An embodiment of embodiment 22, wherein the polylysine comprises ε-poly-L-lysine.

Embodiment 24: An embodiment of embodiment 22 or 23, wherein the concentration of the polylysine in the hydrogel is between 1 mg/mL and 40 mg/mL.

Embodiment 25: An embodiment of any one of embodiments 1-23, wherein the hydrogel further comprises an interpenetrating network of an ionically crosslinked polysaccharide.

Embodiment 26: An embodiment of embodiment 25, wherein the ionically crosslinked polysaccharide comprises calcium crosslinked alginate.

Embodiment 27: An embodiment of embodiment 25 or 26, wherein the interpenetrating network is not bonded to the amine-containing antimicrobial agents.

Embodiment 28: An embodiment of embodiment 1, wherein: the hydrogel further comprises thiolated hyaluronic acid and 4-arm PEG-aldehyde; and the amine-containing antimicrobial agents comprise a plurality of bacteriophages.

Embodiment 29: An embodiment of embodiment 28, wherein the hydrogel further comprises a hyperbranched PEG-acrylate.

Embodiment 30: An embodiment of embodiment 1, wherein: the hydrogel further comprises ε-poly-L-lysine and 4-arm PEG-aldehyde; and the amine-containing antimicrobial agents comprise a plurality of bacteriophages.

Embodiment 31: An embodiment of any one of embodiments 28-30, wherein the hydrogel further comprises 4-arm PEG-benzaldehyde.

Embodiment 32: An embodiment of embodiment 1, wherein the hydrogel further comprises a polymer functionalized with aldehyde groups, with benzaldehyde groups, or with a combination thereof.

Embodiment 33: An embodiment of embodiment 32, wherein the polymer comprises alginate or a derivative thereof.

Embodiment 34: An embodiment of embodiment 32 or 33, wherein the polymer comprises ionically crosslinked alginate.

Embodiment 35: An embodiment of embodiment 32 or 33, wherein the polymer comprises alginate crosslinked with poly-L-lysine.

Embodiment 36: An embodiment of embodiment 32, wherein the polymer comprises hyaluronic acid or a derivative thereof.

Embodiment 37: An embodiment of any one of embodiment 32-36, wherein the mass ratio of the aldehyde groups to the benzaldehyde groups in the hydrogel is at least 5:1.

Embodiment 38: A method of treating an infection in a subject, the method comprising administering to the subject the hydrogel of any one of embodiments 1-37.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only and is not intended to limit the disclosure. In addition, each reference provided herein is incorporated by reference in its entirety for all purposes to the same extent as if each reference was individually incorporated by reference.

What is claimed is:

1. A hydrogel comprising a plurality of amine-containing antimicrobial agents located within the interior of the hydrogel, wherein each of at least a portion of the amine-containing antimicrobial agents is connected to the hydrogel via one or more dynamic covalent imine bonds, and wherein the hydrogel is engineered to facilitate a controlled release of the amine-containing antimicrobial agents from the interior of the hydrogel.

2. The hydrogel of claim 1, wherein the plurality of amine-containing antimicrobial agents comprises a plurality of bacteriophages, a plurality of antibiotics, a plurality of antimicrobial enzymes, a plurality of antimicrobial peptides, or a combination thereof.

3. The hydrogel of claim 2, wherein the plurality of amine-containing antimicrobial agents comprises the plurality of bacteriophages.

4. The hydrogel of claim 3, wherein the concentration of the plurality of bacteriophages in the hydrogel is between 10⁹ PFU/mL and 10¹³ PFU/mL.

5. The hydrogel of claim 1, wherein the hydrogel is crosslinked with dynamic covalent hydrazone bonds, dynamic covalent hemithioacetal bonds, stable covalent thioether bonds, dynamic covalent imine bonds, ionic bonds, static covalent bonds, or a combination thereof.

6. The hydrogel of claim 5, wherein the hydrogel is crosslinked with dynamic covalent hemithioacetal bonds, stable covalent thioether bonds, or a combination thereof.

7. The hydrogel of claim 1, wherein the hydrogel further comprises a thiolated anionic polysaccharide.

8. The hydrogel of claim 7, wherein the thiolated anionic polysaccharide comprises thiolated hyaluronic acid.

9. The hydrogel of claim 7, wherein the concentration of the thiolated anionic polysaccharide in the hydrogel is between 1.5 mg/mL and 15 mg/mL.

10. The hydrogel of claim 1, wherein the hydrogel further comprises an aldehyde-functionalized crosslinker.

11. The hydrogel of claim 10, wherein the aldehyde-functionalized crosslinker comprises a multi-arm poly(ethylene glycol) (PEG)-aldehyde.

12. The hydrogel of claim 11, wherein the multi-arm PEG-aldehyde comprises PEG-dialdehyde, 4-arm PEG-aldehyde, or a combination thereof.

13. The hydrogel of claim 10, wherein the concentration of the aldehyde-functionalized crosslinker in the hydrogel is between 22.5 mg/mL and 225 mg/mL.

14. The hydrogel of claim 1, wherein the hydrogel further comprises a benzaldehyde-functionalized crosslinker.

15. The hydrogel of claim 14, wherein the benzaldehyde-functionalized crosslinker comprises a multi-arm PEG-benzaldehyde.

16. The hydrogel of claim 15, wherein the multi-arm PEG-benzaldehyde comprises PEG-dibenzaldehyde, 4-arm PEG-benzaldehyde, or a combination thereof.

17. The hydrogel of claim 14, wherein the concentration of the benzaldehyde-functionalized crosslinker in the hydrogel is between 2.5 mg/mL and 25 mg/mL.

18. The hydrogel of claim 1, wherein the hydrogel comprises an aldehyde-functionalized crosslinker and a benzaldehyde-functionalized crosslinker, and wherein the mass ratio of the aldehyde-functionalized crosslinker to the benzaldehyde-functionalized crosslinker in the hydrogel is at least 5:1.

19. The hydrogel of claim 1, wherein the hydrogel further comprises an acrylate-functionalized crosslinker.

20. The hydrogel of claim 19, wherein the acrylate-functionalized crosslinker comprises PEG diacrylate, a hyperbranched PEG acrylate, or a combination thereof.

21. The hydrogel of claim 19, wherein the concentration of the acrylate-functionalized crosslinker in the hydrogel is between 0.3 mg/mL and 3 mg/mL.

22. The hydrogel of claim 1, wherein the hydrogel further comprises a polylysine.

23. The hydrogel of claim 22, wherein the polylysine comprises ε-poly-L-lysine.

24. The hydrogel of claim 22, wherein the concentration of the polylysine in the hydrogel is between 1 mg/mL and 40 mg/mL.

25. The hydrogel of claim 1, wherein the hydrogel further comprises an interpenetrating network of an ionically crosslinked polysaccharide.

26. The hydrogel of claim 25, wherein the ionically crosslinked polysaccharide comprises calcium crosslinked alginate.

27. The hydrogel of claims 25, wherein the interpenetrating network is not bonded to the amine-containing antimicrobial agents.

28. The hydrogel of claim 1, wherein:
the hydrogel further comprises thiolated hyaluronic acid and 4-arm PEG-aldehyde; and
the amine-containing antimicrobial agents comprise a plurality of bacteriophages.

29. The hydrogel of claim 28, wherein the hydrogel further comprises a hyperbranched PEG-acrylate.

30. The hydrogel of claim 1, wherein:
the hydrogel further comprises ε-poly-L-lysine and 4-arm PEG-aldehyde; and
the amine-containing antimicrobial agents comprise a plurality of bacteriophages.

31. The hydrogel of claim 28, wherein the hydrogel further comprises 4-arm PEG-benzaldehyde.

32. The hydrogel of claim 1, wherein the hydrogel further comprises a polymer functionalized with aldehyde groups, with benzaldehyde groups, or with a combination thereof.

33. The hydrogel of claim 32, wherein the polymer comprises alginate or a derivative thereof.

34. The hydrogel of claim 32, wherein the polymer comprises ionically crosslinked alginate.

35. The hydrogel of claim 32, wherein the polymer comprises alginate crosslinked with poly-L-lysine.

36. The hydrogel of claim 32, wherein the polymer comprises hyaluronic acid or a derivative thereof.

37. The hydrogel of claim 32, wherein the mass ratio of the aldehyde groups to the benzaldehyde groups in the hydrogel is at least 5:1.

38. A method of treating an infection in a subject, the method comprising administering to the subject the hydrogel of claim 1.
